(19) United States
(12) Patent Application Publication
(10) Pub. No.: US 2008/0293746 A1
(43) Pub. Date: Nov. 27, 2008

(54) COMBINATIONS COMPRISING PREGABALIN

(75) Inventor: Kevin Paul Gunn, Sandwich (GB)

Correspondence Address: PFIZER INC., PATENT DEPARTMENT, MS8260-1611, EASTERN POINT ROAD, GROTON, CT, 06340, US

(73) Assignee: Pfizer, Inc., New York, NY (US)

(21) Appl. No.: 12/112,681

(22) Filed: Apr. 30, 2008

Related U.S. Application Data
(60) Provisional application No. 60/915,174, filed on May 1, 2007.

(51) Int. Cl. A61K 31/519; A61P 29/00

(52) U.S. Cl. 514/262.1

(57) ABSTRACT

The invention relates to a combination of pregabalin and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, to pharmaceutical compositions containing the combination, and to the use of the combination in the treatment of pain.

### CROSS-REFERENCE TO RELATED APPLICATION

This application claims the benefit of U.S. Provisional Application No. 60/915,174 filed May 1, 2007, which application is incorporated by reference herein in its entirety.

### FIELD OF THE INVENTION

The invention relates to a combination of pregabalin and 1-(2-ethoxyethyl)-5-[ethyl(methyl)amino]-N-mesyl-7-[(4-methyl-2-pyridyl)amino]-1H-pyrazolo[4,3-o]pyrimidine-3-carboxamide, to pharmaceutical compositions containing the combination, and to the use of the combination in the treatment of pain.

### BACKGROUND OF THE INVENTION

Pregabalin, (S)-(+)-4-amino-3-(2-methylpropyl)butanoic acid (Lyrica™), an alpha-2-delta ligand, is described in European patent application publication number EP641330A as an anti-convulsant treatment useful in the treatment of epilepsy and in EP0934061A for the treatment of pain.

1-(2-Ethoxyethyl)-5-[ethyl(methyl)amino]-N-mesyl-7-[(4-methyl-2-pyridyl)amino]-1H-pyrazolo[4,3-d]pyrimidine-3-carboxamide, a selective phosphodiesterase-5 (PDE5) inhibitor, is described in WO-A-2005/049616. Crystalline forms of 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide are described in WO-A-2006/120552. 1-(2-Ethoxyethyl)-5-[ethyl(methyl)amino]-N-mesyl-7-[(4-methyl-2-pyridyl)amino]-1H-pyrazolo[4,3-d]pyrimidine-3-carboxamide may exists as two tautomeric isomers, as described in WO-A-2006/120552. The compound I-(2-ethoxyethyl)-5-[ethyl(methyl)amino]-N-mesyl-7-[(4-methyl-2-pyridyl)amino]-1H-pyrazolo[4,3-o]pyrimidine-3-carboxamide is also known as N-[1-(2-ethoxyethyl)-5-(N-ethyl-N-methylamino)-7-(4-methylpyridin-2-yl-amino)-1H-pyrazolo[4,3-d]pyrimidine-3-carbonyl]methanesulfonamide. The nomenclature 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide (or N-[1-(2-ethoxyethyl)-5-(N-ethyl-N-methylamino)-7-(4-methylpyridin-2-yl-amino)-1H-pyrazolo[4,3-d]pyrimidine-3-carbonyl]methanesulfonamide), as used herein, includes all crystalline forms and all tautomeric isomers of the compound, in particular as exemplified by the resonance structures below:

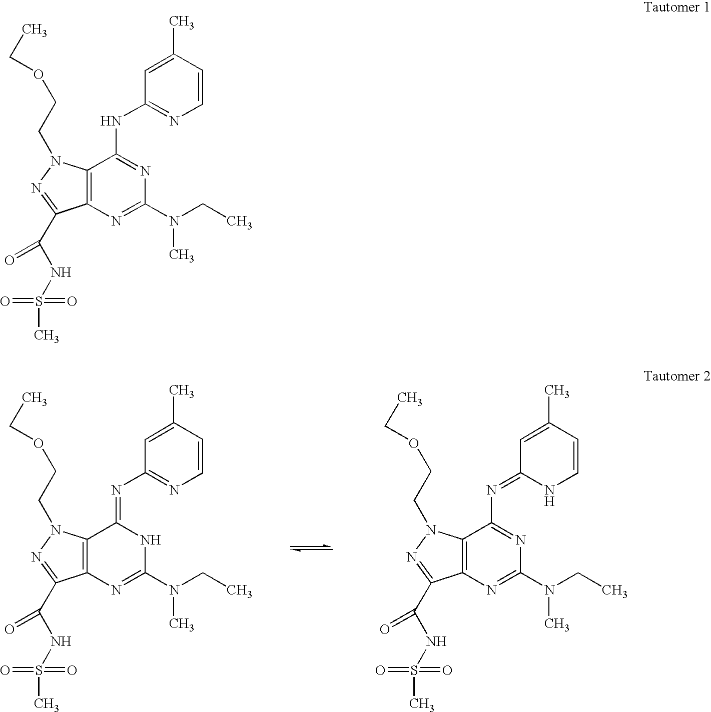

Combinations of alpha-2-delta ligands and PDE5 inhibitors are described in WO-A-2004/016259.

### BRIEF DESCRIPTION OF THE DRAWINGS

FIG. 1 shows the dose response effect of pregabalin on CCI-induced static allodynia.

FIG. 2 shows the dose response effect of 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide on CCI-induced static allodynia.

FIG. 3 shows the effect of a fixed dose ratio combination of pregabalin: 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide on CCI-induced static allodynia.

FIG. 4 shows the effect of various doses of pregabalin in combination with 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide on CCI-induced static allodynia.

FIG. 5 shows the dose response effect of Pregabalin at 2 hrs post administration in the CCI-induced static allodynia rat model in comparison to the effect achieved in the combination with 0.3 mg/kg of 1-(2-ethoxyethyl)-5-[ethyl(methyl)amino]-N-mesyl-7-[(4-methyl-2-pyridyl)amino]-1H-pyrazolo[4,3-o]pyrimidine-3-carboxamide on CCI-induced static allodynia.

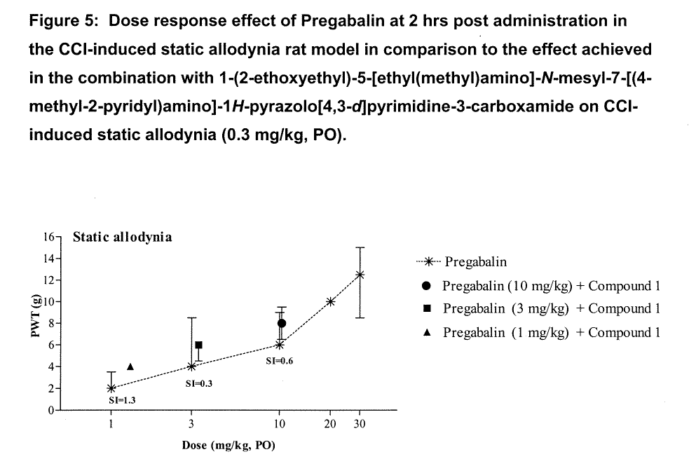

### BRIEF SUMMARY AND DESCRIPTION OF THE INVENTION

It has now been found that combination therapy with pregabalin and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide results in unexpected improvement in the treatment of pain. When administered simultaneously, sequentially or separately, pregabalin and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide interact in a synergistic manner to control pain. This unexpected synergy allows a reduction in the dose required of each compound, leading to a reduction in the side effects and enhancement of the clinical utility of the compounds. The combination is suitable for administration as a twice daily dosage regime, providing an advantage over treatments which require administration three times daily.

Accordingly, the invention provides, as a first aspect, a combination including pregabalin, or a pharmaceutically acceptable salt or solvate thereof, and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, or a pharmaceutically acceptable salt or solvate thereof.

As an alternative or further aspect, the invention provides a pharmaceutical composition including a combination of pregabalin, or a pharmaceutically acceptable salt or solvate thereof, and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, or a pharmaceutically acceptable salt or solvate thereof, and one or more pharmaceutically acceptable excipients.

As an alternative or further aspect, the invention provides a combination of pregabalin or a pharmaceutically acceptable salt or solvate thereof, and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide or a pharmaceutically acceptable salt or solvate thereof, for use as a medicament.

As an alternative aspect, there is provided a method for the curative, prophylactic or palliative treatment of pain, including simultaneous, sequential or separate administration of a therapeutically effective amount of pregabalin, or a pharmaceutically acceptable salt or solvate thereof, and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, or a pharmaceutically acceptable salt or solvate thereof, to a mammal, including a human, in need of said treatment.

As an alternative or further aspect, the invention provides the use of a combination of pregabalin, or a pharmaceutically acceptable salt or solvate thereof, and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, or a pharmaceutically acceptable salt or solvate thereof, in the manufacture of a medicament for the treatment of pain.

As an alternative or further aspect, the invention provides a combination of pregabalin or a pharmaceutically acceptable salt or solvate thereof, and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide or a pharmaceutically acceptable salt or solvate thereof, for use in the treatment of pain.

Suitably the daily dose of pregabalin for use in a human is in a range selected from 1-1000 mg/day, 1-750 mg/day or 50-750 mg/day; more suitably 50-750 mg/day; most suitably 75-600 mg/day. The total daily dose may be administered in single or divided doses.

Suitably the daily dose of 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide for use in a human is in a range selected from 1-300 mg/day, 1-200 mg/day or 1-100 mg/day; more suitably 1-50 mg/day. The total daily dose may be administered in single or divided doses.

The combination according to the present invention may be administered in any suitable relative daily dose range. Suitably the invention includes pregabalin: 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide daily dose range of between 1:1 to 1,000:1 parts by weight. More suitably, the daily dose range is 1:1 to 750:1; more preferably the daily dose range is 1:1 to 600:1; more preferably, the daily dose ratio is of the order of 1:1 to 200:1; more preferably, the daily dose ratio is of the order of 1:1 to 150:1. It will be appreciated that the exact optimum dose ratio will depend on the subject or species to be treated.

As an example of a suitable dosing regimen, pregabalin is administered to a patient at 200 to 400 mg/day, preferably 300 mg/day, in two equal doses (100 to 200 mg, preferably 150 mg in the morning and 100 to 200 mg, preferably 150 mg in the evening), and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide is administered at 5 to 15 mg/day, preferably 10 mg/day as a single dose in the morning. It may be desirable to start at a lower dose and titrate the patient up to the desired therapeutic dose. So, for example, the patient may be administered pregabalin at 150 mg/day in two equal doses, and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide at 4 mg/day for the initial period of the treatment, such as the first 3, 4, 5, 6 or 7 days of treatment, before receiving the full dose of 300 mg/day of pregabalin and 10 mg/day of 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide.

As a further aspect of the present invention, there is provided a combination for human administration comprising pregabalin, or pharmaceutically acceptable salts or solvates thereof, and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, or pharmaceutically acceptable salts or solvates thereof, in a w/w combination range which corresponds to a combination range for pregabalin: 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide of the order of 1:10 to 50:1; 1:10 to 40:1; 1:1 to 40:1; or 1:1 to 30:1 parts by weight in the rat model of chronic constriction injury (CCI) induced static allodynia: more preferably the combination range is of the order 1:1 to 30:1, most preferably 3:1 to 30:1 by weight in the rat model of chronic constriction injury (CCI) induced static allodynia.

For humans, several experimental pain models may be used in man to demonstrate that agents with proven synergy in animals also have effects in man compatible with that synergy. Examples of human models that may be fit for this purpose include the heat/capsaicin model (Petersen, K. L. & Rowbotham, M. C. (1999) NeuroReport 10, 1511-1516), the i.d capsaicin model (Andersen, O. L., Felsby, S., Nicolaisen, L., Bjerring, P., Jsesn, T. S. & Arendt-Nielsen, L. (1996) Pain 66, 51-62), including the use of repeated capsaicin trauma (Witting, N., Svesson, P., Arendt-Nielsen, L. & Jensen, T. S. (2000) Somatosensory Motor Res. 17, 5-12), and summation or wind-up responses (Curatolo, M. et al. (2000) Anesthesiology 93, 1517-1530).

With these models, subjective assessment of pain intensity or areas of hyperalgesia may be used as endpoints, or more objective endpoints, reliant on electrophysiological or imaging technologies (such as functional magnetic resonance imaging) may be employed (Bornhovd, K., Quante, M., Glauche, V., Bromm, B., Weiller, C. & Buchel, C. (2002) Brain 125, 1326-1336). All such models require evidence of objective validation before it can be concluded that they provide evidence in man of supporting the synergistic actions of a combination that have been observed in animal studies.

Doses of each component for synergy can be determined according to published procedures in animal models. However, in man (even in experimental models of pain) the cost can be very high for studies to determine the entire exposure-response relationship at all therapeutically relevant doses of each component of a combination. It may be necessary, at least initially, to estimate whether effects can be observed that are consistent with synergy at doses that have been extrapolated from those that give synergy in animals. In scaling the doses from animals to man, factors such as relative body weight/body surface area, relative absorption, distribution, metabolism and excretion of each component and relative plasma protein binding need to be considered and, for these reasons, the optimal dose ratio predicted for man (and also for patients) is unlikely to be the same as the dose ratio shown to be optimal in animals. However, the relationship between the two can be understood and calculated by one skilled in the art of animal and human pharmacokinetics. Important in establishing the bridge between animal and human effects are the plasma concentrations obtained for each component used in the animal studies, as these are related to the plasma concentration of each component that would be expected to provide efficacy in man. Pharmacokinetic/pharmacodynamic modeling (including methods such as isobolograms, interaction index and response surface modelling) and simulations may help to predict synergistic dose ratios in man, particularly where either or both of these components has already been studied in man.

It is important to ascertain whether any concluded synergy observed in animals or man is due solely to pharmacokinetic interactions. For example, inhibition of the metabolism of one compound by another might give a false impression of pharmacodynamic synergy. In animal studies with pregabalin and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, repeated blood samples have been taken and it has been shown that, in accordance with the known pharmacokinetic properties of the agents, there is no evidence of any pharmacokinetic interaction when the compounds are administered at the doses that induced synergistic pain interactions. This proves that the synergy with respect to pain is pharmacodynamic, occurring subsequent to each of these agents interacting with their respective receptor and/or enzyme targets.

It will be apparent to the skilled reader that the plasma concentration ranges of pregabalin and 1-(2-ethoxyethyl)-5-[ethyl(methyl)amino]-N-mesyl-7-[(4-methyl-2-pyridyl)amino]-1H-pyrazolo[4,3-o]pyrimidine-3-carboxamide required to provide a therapeutic effect depend on the species to be treated, and components used. For example, in combination studies conducted in the rat chronic constriction injury (CCI) model the free plasma concentration of pregabalin ranged from 13 μM to 54 μM at 2 hours 30 minutes post dose and the free plasma concentration of 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide ranged from 0.6 nM to 6.3 nM at 2 hours 30 minutes post dose.

As an alternative aspect, the present invention provides a combination comprising pregabalin, or pharmaceutically acceptable salts or solvates thereof, and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, or pharmaceutically acceptable salts or solvates thereof, wherein the free plasma concentration range ratio for pregabalin: 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide in a human is from is 600:1 to 24,000:1; 600:1 to 12,000:1; or 600:1 to 6,000:1.

The combination of the present invention is suitable for administration as a twice daily dosage regime, providing an advantage over treatments which require administration three times daily. Pregabalin and 1-(2-ethoxyethyl)-5-[ethyl(methyl)amino]-N-mesyl-7-[(4-methyl-2-pyridyl)amino]-1H-pyrazolo[4,3-o]pyrimidine-3-carboxamide may be administered simultaneously, sequentially or separately.

Pregabalin is a marketed product which can be administered twice daily or three times daily in the treatment of neuropathic pain.

The pharmacokinetics of sildenafil after oral tablet administration have been extensively studied in healthy volunteers. The pharmacokinetics of 1-(2-ethoxyethyl)-5-[ethyl(methyl)amino]-N-mesyl-7-[(4-methyl-2-pyridyl)amino]-1H-pyrazolo[4,3-o]pyrimidine-3-carboxamide have been recently studied in several healthy volunteer studies. The estimated terminal half-life (T½) for sildenafil and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide are 4 h and ˜10-15 h, respectively. Therefore, three times a day dosing with oral doses of sildenafil would lead to a steady state plasma concentration time profile with an approximate 4 fold peak to trough ratio. In comparison, based on the measured half-life a similar plasma concentration time profile (similar peak to trough ratio) could be achieved with 1-(2-ethoxyethyl)-5-[ethyl(methyl)amino]-N-mesyl-7-[(4-methyl-2-pyridyl)amino]-1H-pyrazolo[4,3-o]pyrimidine-3-carboxamide administered as a once or twice daily dosage regimen. Thus, a combination product of sildenafil and pregabalin would need to be administered 3 times daily to maintain steady state concentration time profiles with low peak to trough ratio for both compounds. In comparison, a combination product containing 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide and pregabalin could achieve a low peak to trough ratio for both compounds following twice daily administration.

The combination of the invention is potentially useful in the treatment of a range of disorders. The treatment of pain, particularly, neuropathic pain, is a preferred use.

Physiological pain is an important protective mechanism designed to warn of danger from potentially injurious stimuli from the external environment. The system operates through a specific set of primary sensory neurones and is activated by noxious stimuli via peripheral transducing mechanisms (see Millan, 1999, Prog. Neurobiol., 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a hightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibres associated with maladaptation and aberrant activity (Woolf & Salter, 2000, Science, 288, 1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumour related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact etiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

It should be noted that some types of pain have multiple aetiologies and thus can be classified in more than one area, e.g. back pain and cancer pain have both nociceptive and neuropathic components.

Other types of pain include:
pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis;
heart and vascular pain, including pain caused by angina, myocardical infarction, mitral stenosis, pericarditis, Raynaud's phenomenon, scleredoma and skeletal muscle ischemia;
head pain, such as migraine (including migraine with aura and migraine without aura), cluster headache, tension-type headache mixed headache and headache associated with vascular disorders; and
orofacial pain, including dental pain, otic pain, burning mouth syndrome and temporomandibular myofascial pain.

Pharmaceutically acceptable salts of the components of the combination include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of the components of the invention may be prepared by one or more of three methods:

(i) by reacting the compound with the desired acid or base;
(ii) by removing an acid- or base-labile protecting group from a suitable precursor of the compound or by ring-opening a suitable cyclic precursor, for example, a lactone or lactam, using the desired acid or base; or
(iii) by converting one salt of the compound to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The components of the combination of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The components of the combination of the invention may also exist in unsolvated and solvated forms. The term ‘solvate’ is used herein to describe a molecular complex comprising the component of the combination and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The components of the combination of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘Iyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to pregabalin and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide include references to salts, solvates, multi-component complexes and liquid crystals thereof and to solvates, multi-component complexes and liquid crystals of salts thereof.

The components of the combination of the invention include pregabalin and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide as hereinbefore defined, including all polymorphs and crystal habits thereof, prodrugs and isomers thereof (including optical, geometric and tautomeric isomers) as hereinafter defined and isotopically-labeled pregabalin and 1-[(2-ethoxyethyl)-5 ethyl(methyl)amino]-N-mesyl-7-[(4-methyl-2-pyridyl)amino]-1H-pyrazolo[4,3-d]pyrimidine-3-carboxamide.

As indicated, so-called ‘prodrugs’ of the components of the combination are also within the scope of the invention. Thus certain derivatives of pregabalin or 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide which may have little or no pharmacological activity themselves can, when administered into or onto the body, be converted into pregabalin or 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in pregabalin and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide with certain moieties known to those skilled in the art as ‘pro-moieties’ as described, for example, in Design of Prodrugs by H. Bundgaard (Elsevier, 1985).

Some examples of prodrugs in accordance with the invention include

(i) where the compound contains a carboxylic acid functionality (—COOH), an ester thereof, for example, a compound wherein the hydrogen of the carboxylic acid functionality of the compound is replaced by (C₁-C₈)alkyl; and
(ii) where the compound contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl.

Further examples of replacement groups in accordance with the foregoing examples and examples of other prodrug types may be found in the aforementioned references.

Also included within the scope of the invention are metabolites of the components of the combination of the invention, that is, compounds formed in vivo upon administration of the drug. Some examples of metabolites in accordance with the invention include

(i) where the compound contains a methyl group, an hydroxymethyl derivative thereof (—CH₃->—CH₂OH):
(ii) where the compound contains an alkoxy group, an hydroxy derivative thereof (—OR->—OH);
(iii) where the compound contains a tertiary amino group, a secondary amino derivative thereof (—NR¹R²->—NHR¹ or —NHR²);
(iv) where the compound contains a secondary amino group, a primary derivative thereof (—NHR¹->—NH₂); and
(vi) where the compound contains a carboxamide group, a carboxylic acid derivative thereof (—CONH₂->COOH).

The compound I-(2-ethoxyethyl)-5-[ethyl(methyl)amino]-N-mesyl-7-[(4-methyl-2-pyridyl)amino]-1H-pyrazolo[4,3-d]pyrimidine-3-carboxamide (also known as N-[1-(2-ethoxyethyl)-5-(N-ethyl-N-methylamino)-7-(4-methylpyridin-2-yl-amino)-1H-pyrazolo[4,3-d]pyrimidine-3-carbonyl]methanesulfonamide) may exist as structural isomers which are interconvertible via a low energy barrier, i.e. tautomeric isomerism (‘tautomerism’) can occur. All tautomeric forms of the compound are suitable for use in the combination of the invention.

The present invention includes all pharmaceutically acceptable isotopically-labelled components of the combination wherein one or more atoms are replaced by atoms having the same atomic number, but an atomic mass or mass number different from the atomic mass or mass number which predominates in nature.

Examples of isotopes suitable for inclusion in the components of the combination of the invention include isotopes of hydrogen, such as ²H and ³H, carbon, such as ¹¹C, ¹³C and ¹⁴C, nitrogen, such as ¹³N and ¹⁵N, oxygen, such as ¹⁵O, ¹⁷O and ¹⁸O, and sulphur, such as ³⁵S.

Certain isotopically-labelled components of the combination, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁵O and ¹³N, can be useful. in Positron Emission Topography (PET) studies for examining substrate receptor occupancy.

Isotopically-labeled compounds can generally be prepared by conventional techniques known to those skilled in the art

Pharmaceutically acceptable solvates in accordance with the invention include those wherein the solvent of crystallization may be isotopically substituted, e.g. D₂O, d₆-acetone, d₆-DMSO.

The components of the combination of the invention should be assessed for their biopharmaceutical properties, such as solubility and solution stability (across pH), permeability, etc., in order to select the most appropriate dosage form and route of administration for treatment of the proposed indication.

The components of the combination of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

The components of the combination of the instant invention may be administered separately, simultaneously or sequentially. The combination of the invention may be administered alone or in a further combination with one or more other drugs (or as any combination thereof). Generally, the components of the combination of the invention will be administered as a formulation in association with one or more pharmaceutically acceptable excipients. The term ‘excipient’ is used herein to describe any ingredient other than the components of the combination of the invention. The choice of excipient will to a large extent depend on factors such as the particular mode of administration, the effect of the excipient on solubility and stability, and the nature of the dosage form.

Pharmaceutical compositions suitable for the delivery of combination of the present invention and methods for their preparation will be readily apparent to those skilled in the art. Such compositions and methods for their preparation may be found, for example, in Remington's Pharmaceutical Sciences, 19th Edition (Mack Publishing Company, 1995).

### Oral Administration

The components of the combination of the invention may be administered orally. Oral administration may involve swallowing, so that the components of the combination enter the gastrointestinal tract, and/or buccal, lingual, or sublingual administration by which the components enter the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The components of the combination of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a component of the combination, a film-forming polymer, a binder, a solvent, a humectant, a plasticiser, a stabiliser or emulsifier, a viscosity-modifying agent and a solvent. Some components of the formulation may perform more than one function.

The components of the combination may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the components of the combination may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

### Parenteral Administration

The components of the combination of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of the components of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus the components of the combination may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic) acid (PGLA) microspheres.

### Topical Administration

The components of the combination of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

### Inhaled/Intranasal Administration

The components of the combination of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the component(s) of the combination of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticies, high pressure homogenisation, or spray drying.

Capsules (made, for example, from gelatin or hydroxypropylmethylcellulose), blisters and cartridges for use in an inhaler or insufflator may be formulated to contain a powder mix of the component of the combination of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the component of the combination per actuation and the actuation volume may vary from 1 μl to 100 μl. A typical formulation may comprise the component of the combination, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff”.

### Rectal/Intravaginal Administration

The components of the combination of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

### Ocular/Aural Administration

The components of the combination of the invention may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

### Other Technologies

The components of the combination of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

### Dosage

For administration to human patients, the total daily dose of the components of the combination of the invention may be administered in single or divided doses.

For the avoidance of doubt, references herein to “treatment” include references to curative, palliative and prophylactic treatment.

### Kit-of-Parts

Inasmuch as it may desirable to administer a combination of active compounds, for example, for the purpose of treating a particular disease or condition, it is within the scope of the present invention that two or more pharmaceutical compositions, at least one of which contains a component of the combination in accordance with the invention, may conveniently be combined in the form of a kit suitable for coadministration of the compositions.

Thus the kit of the invention comprises two or more separate pharmaceutical compositions, at least one of which contains a component of the combination in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

The components of the combination of the instant invention may be administered separately, simultaneously or sequentially. The combination may also optionally be administered simultaneously, sequentially or separately in combination with one or more agents selected from:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, oisalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-o]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504);
a sodium channel blocker, such as lidocaine;
a 5-HT3 antagonist, such as ondansetron;
   and the pharmaceutically acceptable salts and solvates thereof.

### BIOLOGY EXAMPLES

### Methods

Animals: Male Sprague Dawley rats (150-250 g at the time of surgery), obtained from Charles River, (Manston, Kent, U.K.) were housed in groups of 3. All animals were kept under a 12 hour light/dark cycle (lights on at 07 h 00 min) with food and water ad libitum. All experiments were carried out by an observer blind to the treatments and in accordance with the Home Office Animals (Scientific Procedures) Act 1986.

### Chronic Constriction Injury (CCI) rat Model of Neuropathic Pain

The CCI of sciatic nerve was performed as previously described by Bennett and Xie (Bennett G J, Xie Y K. A peripheral mononeuropathy in rat that produces disorders of pain sensation like those seen in man. Pain: 33:87-107, 1988). Animals were anaesthetised with a 2% isofluorane/O2 mixture. The right hind thigh was shaved and swabbed with 1% iodine. Animals were then transferred to a homeothermic blanket for the duration of the procedure and anaesthesia maintained during surgery via a nose cone. The skin was cut along the line of the thighbone and the common sciatic nerve was exposed Four ligatures (4-0 silk) were tied loosely around the nerve, 1 cm proximally to the trifurcation, with approx 1 mm spacing. The incision was closed in layers.

### Assessment of Static Allodynia

Animals were habituated to wire bottom test cages prior to the assessment of allodynia. Static allodynia was evaluated by application of calibrated von Frey hairs (Stoelting, Wood Dale, Ill., USA.) in ascending order of force (0.8, 1, 1.4, 2, 4, 6, 8, 10, 15 and 26 grams) to the plantar surface of hind paws. Each von Frey hair was applied to the paw for a maximum of 6 seconds, or until a withdrawal response occurred. Once a withdrawal response to a von Frey hair was established, the paw was re-tested, starting with the filament below the one that produced a withdrawal, and subsequently with the remaining filaments in descending force sequence until no withdrawal occurred. The highest force of 26 g lifted the paw as well as eliciting a response, thus represented the cut off point. Each animal had both hind paws tested in this manner. The lowest amount of force required to elicit a response was recorded as paw withdrawal threshold (PWT) in grams. Static allodynia was defined as present if animals responded to a stimulus of, or less than, 4 g, which is innocuous in naive rats (Field M J, Bramwell S, Hughes J, Singh L. Detection of static and dynamic components of mechanical allodynia in rat models of neuropathic pain: are they signalled by distinct primary sensory neurones? Pain, 1999; 83:303-11).

The effect on the CCI model of neuropathic pain has been examined for pregabalin and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide alone, and for the combination of the present invention.

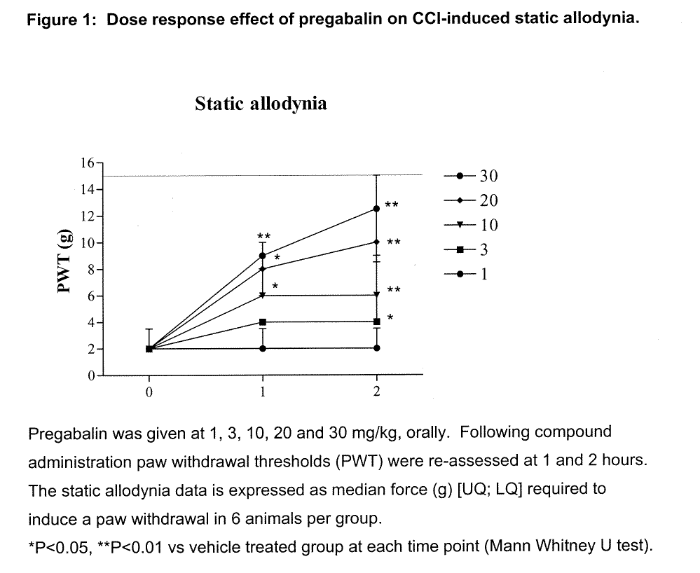

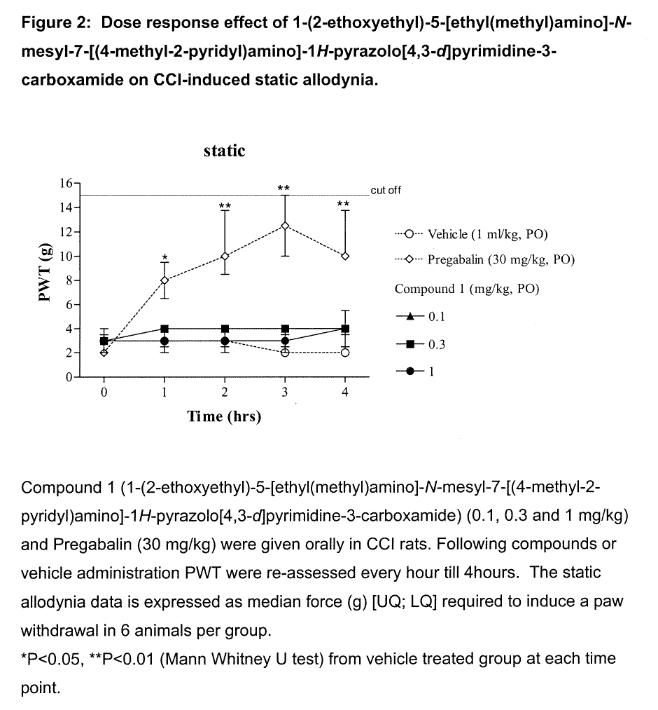

Pregabalin demonstrated robust effects in this model (FIG. 1), with a minimum effective dose (MED) following oral dosing at 3 mg/kg (mean free plasma concentration of 17.8 μM free at 2.5 h after dosing) and a full effect achieved at 20 mg/kg (mean free plasma concentration of 89.8 μM at 2.5 h after dosing). A similar study was completed with 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide with oral dosing at 0.1 to 1 mg/kg (FIG. 2) with no effect observed on any allodynia endpoint (mean free plasma concentrations up to 10.2 nM after 1.3 hours, and 16.1 nM at 4.3 hours for the highest dose tested).

Two further studies examined the effect of the combination of the invention. For these studies a “synergy index (SI)” was calculated for each experiment based on literature precedence, in which a value <1 indicates a statistically relevant synergy has occurred (Berenbaum, M. C. Synergy, Additivism, and antagonism in immunosuppression. A critical review. Clinical and Experimental immunology. 1977; 28:1-18.).

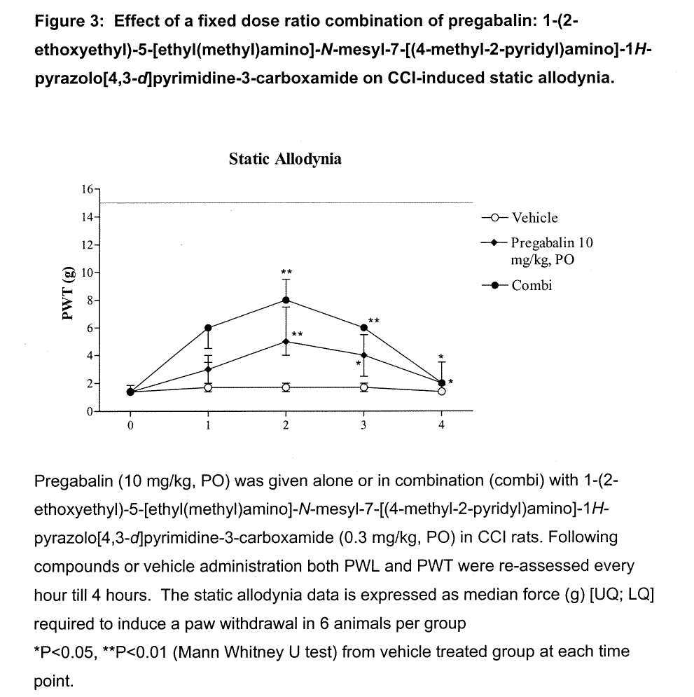

In the first study, a combination of 10 mg/kg pregabalin and 0.3 mg/kg 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide (Synergy Index 0.6) was studied and compared with 10 mg/kg of pregabalin alone (FIG. 3). In this study, pregabalin (mean free plasma exposure of 54 μM achieved) alone 2 hours after dosing partially reversed static allodynia endpoints. The combination of the invention (mean free plasma exposure of 48 μM pregabalin and 4.7 nM 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide) fully reversed static allodynia, indicating an increased effect if compared to pregabalin alone. For this study a synergistic index of 0.6 was calculated confirming synergistic interaction between the agents.

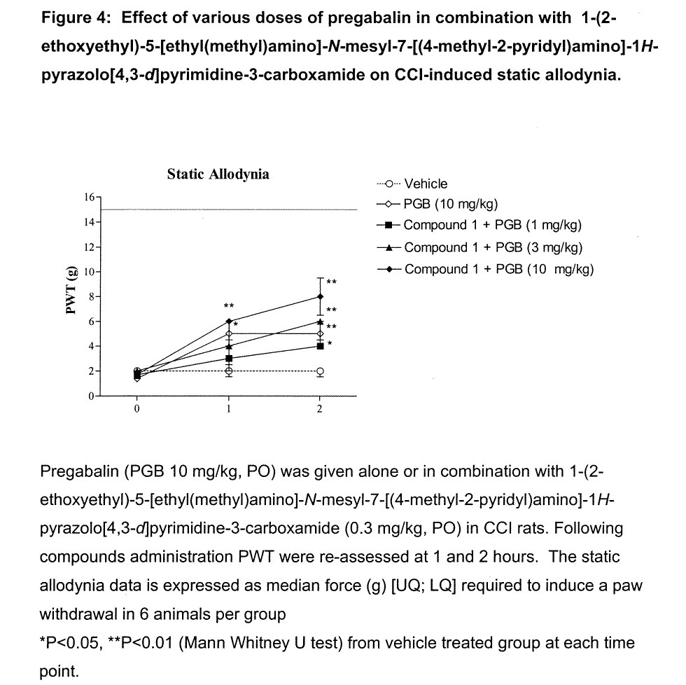

An additional study examined the dose relationship of pregabalin (1, 3, and 10 mg/kg) with a fixed dose of 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide (0.3 mg/kg). A group of rats was orally treated with pregabalin 10 mg/kg to function as positive control in the study (FIG. 4). The combinations were compared with a dose response of pregabalin. The combination demonstrated synergy in static allodynia endpoints. A full reversal of static allodynia was observed with the 0.3 mg/kg of 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide and 10 mg/kg of pregabalin (Synergy Index 0.6); and a partial, but significant, reversal of static allodynia was also observed with the 0.3 mg/kg 1-(2-ethoxyethyl)-5-[ethyl(methyl)amino]-N-mesyl-7-[(4-methyl-2-pyridyl)amino]-1H-pyrazolo[4,3-o]pyrimidine-3-carboxamide and 3 mg/kg of pregabalin (Synergy Index 0.3).

What is claimed is:

1. A combination comprising pregabalin, or a pharmaceutically acceptable salt thereof, and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, or a pharmaceutically acceptable salt thereof.

2. The combination of claim 1 for use as a medicament.

3. The combination of claim 1 for use in the treatment of pain.

4. A pharmaceutical composition comprising pregabalin, or a pharmaceutically acceptable salt thereof; 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, or a pharmaceutically acceptable salt thereof; and one or more pharmaceutically acceptable excipients.

5. A method for the treatment of pain comprising administering a therapeutically effective amount of pregabalin, or a pharmaceutically acceptable salt thereof, and 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, or a pharmaceutically acceptable salt thereof, to a mammal in need of said treatment.

6. The method of claim 5 wherein the mammal is a human.

7. The method of claim 5 wherein the pregabalin, or pharmaceutically acceptable salt thereof, and the 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, or pharmaceutically acceptable salt thereof, are administered simultaneously to the mammal.

8. The method of claim 5 wherein the pregabalin, or pharmaceutically acceptable salt thereof, and the 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, or pharmaceutically acceptable salt thereof, are administered sequentially to the mammal.

9. The method of claim 5 wherein the pregabalin, or pharmaceutically acceptable salt thereof, and the 1-(2-ethoxyethyl)-5-[ethyl(methyl)amino]-N-mesyl-7-[(4-methyl-2-pyridyl)amino]-1H-pyrazolo[4,3-d]pyrimidine-3-carboxamide, or pharmaceutically acceptable salt thereof, are administered separately to the mammal.
